(19) United States
(12) Patent Application Publication
(10) Pub. No.: US 2023/0082709 A1
(43) Pub. Date: Mar. 16, 2023

(54) Aerosol Purifier for HVAC System

(71) Applicant: Trane International Inc., Davidson, NC (US)

(72) Inventors: Zubin Varghese, Bangalore (IN); Rohith L. Balegundi, Bangalore (IN); Yogesh Pandit, Bengaluru (IN); Sandeep Vegeeshwara, Bangalore (IN); Rasha Hasaneen, Mooresville, NC (US); Joel Patrick Gouker, Fort Mill, SC (US); Jennie Bergman, Shoreview, MN (US); Ronald Maurice Cosby, II, La Crosse, WI (US)

(21) Appl. No.: 17/659,438

(22) Filed: Apr. 15, 2022

Related U.S. Application Data
(63) Continuation of application No. 17/456,797, filed on Nov. 29, 2021.

(30) Foreign Application Priority Data
Sep. 15, 2021 (IN) 2021131041607

(51) Int. Cl. F24F 8/30; A61L 9/14; A61L 9/20; A61L 9/22; F24F 13/22

(52) U.S. Cl. CPC F24F 8/30; A61L 9/14; A61L 9/20; A61L 9/22; F24F 13/222; A61L 2209/14; A61L 2209/132; A61L 2209/12; A61L 2209/16; A61L 2209/111

(57) ABSTRACT

An air purifying device, system and method for cleaning and infecting a conditioned space or zone. The air purifying system may include a heating, ventilation and air conditioning (HVAC) system with HVAC equipment configured to provide conditioned air to a conditioned space, where the HVAC system also includes a condensate trap configured to collect condensate produced during operation. The air purifying system also including an air purifying device configured to energize the condensate from the condensate trap to generate an aerosol including negative ions, which are combined the conditioned air provided by the HVAC system to the conditioned space.

### CROSS-REFERENCE TO RELATED APPLICATION

The present application is a continuation of United Stated Patent Office Application No. 17/456797, which was filed on Nov. 29, 2021, by Varghese, et al., and entitled “Aerosol Purifier for HVAC System,” which claims priority to a foreign filing in the Republic of India on Sep. 15, 2021, India Patent Office Application No. 2021131041607, by Varghese, et al., entitled “Aerosol Purifier for HVAC System,” both of which are hereby incorporated by reference in their entirety.

### TECHNOLOGICAL FIELD

The present disclosure relates generally to an aerosol purifier which may be used with a heating, ventilation, and/or air conditioning (HVAC) systems for supplying an aerosol with negative ions to a conditioned space, ensuring good air quality and purifying the space.

### BACKGROUND

Climate control systems, such as heating, ventilation, and/or air conditioning (HVAC) systems are used in residential, industrial, and commercial buildings to heat, cool or otherwise condition spaces by providing conditioned air. These systems often include various components or devices for cleaning air circulated through the device. However, these existing solutions have various limitations, particularly for smaller contaminants and surfaces within the room.

Solid particle air filters such as HEPA filters clean recirculated air and provide acceptable cleaning for many circumstances, however these filters are often limited in terms of the size particles that may be filters. In addition, they generate static pressure losses within the HVAC system, which leads to energy losses. As a result, there is an inverse relationship between particle size/cleaning efficacy and energy efficiency. These filters are also passive elements and, thus, are limited to removing particles from the circulating air prior to entering a space. They are not capable of actively generating cleaning solutions that may be delivered by the HVAC system to the space.

Moreover, existing active cleaning technologies used in the HVAC systems are limited and suffers various drawbacks. For example, electrical field chargers typically use a corona discharge to create an electric field and ionize particles in the air. Given the high power levels and the varying types of contaminates within the air stream, these systems often generate undesired levels of ozone. Similarly, chemical cleaners often generate various cleaning solutions such as hydrogen peroxide, however, these solutions may have undesirable side effects, including the creation of ozone.

Outside the HVAC industry, technologies exist that are capable of generating ions using water. These technologies may utilize water shearing or the Lenard effect, in which electrical charges are generated when kinetic forces break up water into droplets. These droplets may collide with each other to generate ions, which may be used for cleaning in some instances. However, to date, none of these systems utilizes an air purifying device coupled to a HVAC device, particularly not one that utilizes discharge condensate from the HVAC system to generate the cleaning aerosol. Thus, the system described herein has various advantages over the existing technologies.

### BRIEF SUMMARY

Example implementations of the present disclosure provide an air purifying system and associated methods for use with an HVAC system that generates cleaner air for a conditioned space. These air purifying devices, in some examples, use condensate generated by various components of the HVAC system to generate an aerosol to clean the air. The condensate is routed to the aerosol generator, which energizes the water to create an aerosol containing negative ions. This aerosol mixes with the conditioned air and is routed to a conditioned space (and potentially an outside environment), often cleaning both the air and surfaces within the space. Additional control circuitry is also used in some instances to provide enhanced control and operation of the device and system.

The present disclosure thus includes, without limitation, the following example implementations.

Some example implementations provide an air purifying device for a heating, ventilation and air conditioning (HVAC) system operable to provide conditioned air to a conditioned space, the HVAC system including a condensate trap configured to collect condensate produced during operation, the air purifying device comprising: an aerosol generator; and a conduit operably coupleable to and between the condensate trap and the aerosol generator, the conduit configured to deliver the condensate from the condensate trap to the aerosol generator that is configured to energize the condensate to generate an aerosol including negative ions combinable with the conditioned air provided by the HVAC system to the conditioned space.

Some example implementations provide an air purifying system comprising: a heating, ventilation and air conditioning (HVAC) system with HVAC equipment configured to provide conditioned air to a conditioned space, the HVAC system including a condensate trap configured to collect condensate produced during operation; and an air purifying device configured to energize the condensate from the condensate trap to generate an aerosol including negative ions, the aerosol being combinable with the conditioned air provided by the HVAC system to the conditioned space.

Some example implementations provide a method of disinfecting a conditioned space to which heating, ventilation, and air conditioning (HVAC) equipment of an HVAC system is configured to provide conditioned air, the HVAC system including a condensate trap configured to collect condensate produced during operation, the method comprising: delivering the condensate from the condensate trap to an aerosol generator; energizing the condensate at the aerosol generator to generate an aerosol including negative ions; combining the aerosol including the negative ions with the conditioned air provided by the HVAC system; and delivering the combined air and aerosol to the conditioned space.

These and other features, aspects, and advantages of the present disclosure will be apparent from a reading of the following detailed description together with the accompanying figures, which are briefly described below. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example implementations, should be viewed as combinable unless the context of the disclosure clearly dictates otherwise.

It will therefore be appreciated that this Brief Summary is provided merely for purposes of summarizing some example implementations so as to provide a basic understanding of some aspects of the disclosure. Accordingly, it will be appreciated that the above described example implementations are merely examples and should not be construed to narrow the scope or spirit of the disclosure in any way. Other example implementations, aspects and advantages will become apparent from the following detailed description taken in conjunction with the accompanying figures which illustrate, by way of example, the principles of some described example implementations.

### BRIEF DESCRIPTION OF THE FIGURE(S)

Having thus described example implementations of the disclosure in general terms, reference will now be made to the accompanying figures, which are not necessarily drawn to scale, and wherein:

FIG. 1A is a block diagram of an air purifying system coupled to a heating, ventilation, and air conditioning (HVAC) system according to some example implementations of the present disclosure;

FIG. 1B is a block diagram of an air purifying system with an aromatic dispenser coupled to a heating, ventilation, and air conditioning (HVAC) system according to some example implementations of the present disclosure;

FIG. 2A is an illustration of an air handler unit with an air purifying device, according to some example implementations;

FIG. 2B is another illustration of an air handler unit with an air purifying device, according to some example implementations;

FIG. 3 is an illustration of an air purifying device, according to some example implementations;

FIG. 4 is an illustration of a condensate trap, according to some example implementations;

FIG. 5A is an illustration of an aromatic dispenser, according to some example implementations;

FIG. 5B is an illustration of various components of an aromatic dispenser, according to some example implementations;

FIGS. 6A, 6B, 6C, 6D, 6E, 6F, 6G, 6H, and 6I are flowcharts illustrating various operations in a method of controlling an air purifying system, according to some example implementations;

FIGS. 7A, 7B, 7C, 7D, 7E, 7F, 7G, 7H, and 7I are flowcharts illustrating various operations in a method of controlling an air purifying system, according to some example implementations; and

FIG. 8 illustrates control circuitry according to some example implementations.

### DETAILED DESCRIPTION

Some implementations of the present disclosure will now be described more fully hereinafter with reference to the accompanying figures, in which some, but not all implementations of the disclosure are shown. Indeed, various implementations of the disclosure may be embodied in many different forms and should not be construed as limited to the implementations set forth herein; rather, these example implementations are provided so that this disclosure will be thorough and complete, and will fully convey the scope of the disclosure to those skilled in the art. Like reference numerals refer to like elements throughout.

Unless specified otherwise or clear from context, references to first, second or the like should not be construed to imply a particular order. A feature described as being above another feature (unless specified otherwise or clear from context) may instead be below, and vice versa; and similarly, features described as being to the left of another feature may instead be to the right, and vice versa. Also, while reference may be made herein to quantitative measures, values, geometric relationships or the like, unless otherwise stated, any one or more if not all of these may be absolute or approximate to account for acceptable variations that may occur, such as those due to engineering tolerances or the like.

As used herein, unless specified otherwise or clear from context, the “or” of a set of operands is the “inclusive or” and thereby true if and only if one or more of the operands is true, as opposed to the “exclusive or” which is false when all of the operands are true. Thus, for example, “[A] or [B]” is true if [A] is true, or if [B] is true, or if both [A] and [B] are true. Further, the articles “a” and “an” mean “one or more,” unless specified otherwise or clear from context to be directed to a singular form. Furthermore, it should be understood that unless otherwise specified, the terms “data,” “content,” “digital content,” “information,” “observation” and similar terms may be at times used interchangeably.

Example implementations of the present disclosure provide an air purifying device for use with an HVAC system and associated methods for controlling the components therein. The air purifying device may be coupled to a condensate producing component within the HVAC system where the condensate is collected by a condensate trap and routed to an aerosol generator. In some instances, the condensate is purified and/or disinfected prior to entering the aerosol generator. The aerosol generator energizes the condensate, creating an aerosol, which often is composed of water vapor. negative ions are also created from the condensate as part of this process, and the aerosol with the negative ions are combined with conditioned air and routed to a conditioned space. The aerosol and the negative ions may clean the conditioned air as well as the conditioned space, and in some instances, they may also clean surfaces within the space, potentially prior to the space being occupied.

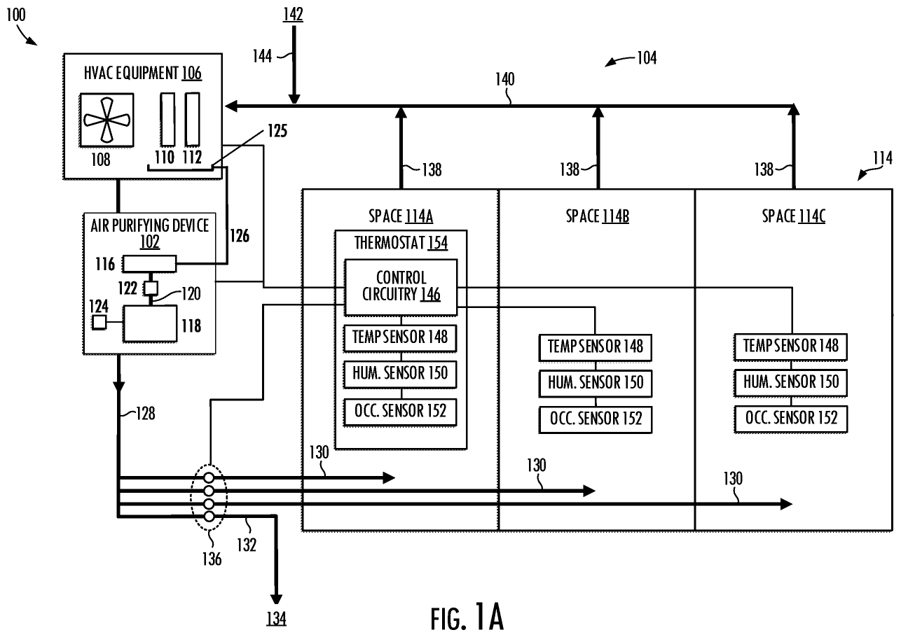

FIG. 1A is a block diagram of the air purifying system 100 according to some example implementations of the present disclosure. This system includes an air purifying device 102 coupled to an HVAC system 104 that generally includes HVAC equipment 106 with a fan 108, cooling element 110, and/or heating elements 112. The cooling element and/or heating element may comprise a heat exchanger coil. The fan 108 may be an indoor fan or blower configured to provide conditioned air to a conditioned space 114, which in some examples may be divided into a plurality of spaces or zones 114A, 114B, 114C (each of which may itself be a conditioned space). It will be appreciated that while three spaces are shown, any number of spaces or zones may be present. It will also be appreciated that references to the conditioned space may be equally applicable to one or more spaces, and/or zones each of which may be comprised of one or more spaces.

The air purifying device 102 may include a condensate trap 116, an aerosol generator 118, a conduit 120, a condensate pump 122, and a drive circuit 124. The condensate trap collects condensate produced by a condensate producing component. Often this component is the cooling element 110, particularly a cooling coil, but other components within the HVAC system may also produce condensate that may be collected by the condensate trap and used by the air purifying device. In some examples, a drain pan 125 is positioned under the condensate producing component and that pan is coupled to a pipe 126 that routes the condensate to the condensate trap as shown in FIG. 1A. Other routing structures may be used. For example, often the condensate trap is located below the condensate producing component and collects the condensate directly.

Once collected, the condensate is routed to the aerosol generator 118 via the conduit 120. In some instances, a condensate pump 122 is used to move the condensate between the condensate trap and the aerosol generator. The condensate pump pressurizes the condensate fluid, directing it through the conduit, vertically if necessary. This pressurization may also allow the condensate to be dispersed within the aerosol generator via a sprayer (not shown) or other fitting, which may aid in the generation of aerosol from the condensate.

The drive circuit 124 is used to energize the aerosol generator, which creates an aerosol with negative ions from the condensate. The aerosol is mixed with the conditioned air and directed to a conditioned space 114. Often the aerosol is mixed with the conditioned air after the conditioned air has been through various HVAC equipment 106, allowing the aerosol to bypass some or all of the HVAC components and be routed directly to the conditioned space.

The HVAC equipment 106 may include an indoor unit, an outdoor unit, and a refrigerant loop extending between the indoor unit and the outdoor unit. The indoor unit may include a furnace or air handler, an indoor refrigerant heat exchanger or evaporator to condition air (heat or cool), and the fan 108 to circulate or otherwise provide the conditioned air to the conditioned space. The outdoor unit may include an outdoor fan and an outdoor refrigerant heat exchanger or condenser, and the refrigerant loop may extend between the indoor and outdoor refrigerant heat exchangers. In more complex systems, often used in higher load applications such as commercial buildings, additional equipment such as chillers, cooling towers, water source heat pumps, etc. may also be included within the HVAC system.

The HVAC equipment 106 may further include a cooling element 110, such as cooling coils used to cool and condition the air provided to the various zones. In some examples, the system utilizes refrigerant cooling coils, and provides a direct exchange of heat between the conditioned air and the refrigerant. Other cooling elements or fluids may also be utilized, including hydronic/water coils, etc. Similarly, the HVAC system 104 may utilize a variety of different heating elements 112, such as electric, gas, refrigerant heated coils, hydronic/water heated coils, etc. In some examples, the cooling element 110 and the heating element 112 may be the same coil within an air handler unit or a heat pump system. The HVAC equipment may be arranged to provide staged capacity relief. In some examples, multiple cooling and/or heating elements are provided to allow the HVAC system to provide staged capacity to the system in either cooling or heating mode. In some examples, the fan is similarly configured to operate at set stages to provide staged capacity to the zones calling for conditioning relief. In other examples, the cooling element, heating element, and/or fan may be continuously adjusted over a set range or range(s) allowing the system to adjust the capacity provided in a continuous or substantially continuous manner over that range.

In some examples, the HVAC system 104 may include an air circulation path with supply air ducts including a main supply air duct 128 and branch supply air ducts 130 through which the conditioned air from the HVAC equipment 106 is provided to the conditioned spaces 114A, 114B, 114C. As shown in FIG. 1A, in some examples, the air purifying device 102 is coupled to the main supply air duct, allowing the aerosol generated from the condensate to combine with conditioned air within the supply air duct. In addition, in some instances, the HVAC system includes a branch supply duct 132 directing air to an outside environment 134. To control airflow to the zones, the HVAC equipment may include zone dampers 136. The air circulation path also includes return air ducts including space return air ducts 138 and a main return air duct 140 operably coupleable with the HVAC equipment, and through which air in the zones of the conditioned space is returned to the HVAC equipment as return air. In some examples, the HVAC system includes a supply of outdoor air 142 directed through a return air duct 144, and in some examples, this outdoor air is coupled to the main return air duct 140.

The HVAC system 104 in FIG. 1A includes control circuitry 146 operably coupled with the HVAC equipment 106 and the air purifying device 102. The control circuitry is configured to control the air purifying device, adjusting both the volume and type of aerosol produced. The control circuitry is also configured to control the HVAC equipment to provide the conditioned air to heat or cool the conditioned space 114, or in some examples, spaces 114A, 114B, 114C of the conditioned space. In some examples, the control circuitry is configured to control the air purifying device and/or the HVAC equipment based on environmental feedback from one or more sensors operably coupled to the control circuitry. In this regard, the HVAC system may include at least one of a temperature sensor 148, a humidity sensor 150, and an occupancy sensor 152, or potentially other sensors such as an air quality sensor.

In some examples, the HVAC system 104 may include a conditioned air temperature sensor 148 in the conditioned space 114 configured to measure temperature in the conditioned space. In some examples, as shown, one or more of the spaces 114A, 114B, 114C may include a respective conditioned air temperature sensor for the space and configured to measure temperature in the space. In some examples, the control circuitry 146 (or some portion of the control circuitry) and the conditioned air temperature sensor may be co-located in a thermostat 154. In other examples, the control circuitry and the conditioned air temperature sensor may be separated and connected by wired or wireless communication; and in some of these examples, the conditioned air temperature sensor may be co-located with other electronic devices or circuitry such as a display device, network adapter, or the like.

Similarly, the humidity sensor 150 and the occupancy sensor 152 may be co-located within a central device such a thermostat 154. In other examples, some or all of these sensors may be located as separate components or part of another device. The ooccupancy sensors may provide an indication of the number of people within a space. These occupancy sensors may be any type of sensor that provides this functionality including, carbon dioxide (CO2) sensors, motion sensors, video fields, thermal imaging cameras, temperatures sensors, or others, some of which may be combined with analytics to provide more accurate occupancy information. Humidity sensors 150 may also be used to provide humidity information, e.g., relative humidity, web bulb temperature, etc. The control circuitry may further be coupled to auxiliary devices, such as operating schedules, occupancy preference, real-time clocks, energy rate schedules, etc.

FIG. 1B shows an example embodiment where the system discussed above in regard to FIG. 1A that also includes an aromatic dispenser 160 that dispenses an aromatic aerosol which may be combined with the conditioned air and delivered to the conditioned space(s). The aromatic dispenser may include a dispenser 162 that dispenses an aromatic 164 that is aerosolized by an aromatic aerosol generator 166.

The aerosol dispenser 160 may include a dispenser 162 that dispenses an aromatic 164. The aromatic may be combined with a fluid to create an aromatic fluid. The aromatic fluid is then supplied to an aromatic aerosol generator 166 that produces an aromatic aerosol that may be combined with the conditioned air provided by the HVAC system for delivery to the conditioned space.

In some examples, the HVAC system 104 includes control circuitry 146 operably coupled with the HVAC equipment 106, the air purifying device 102, and the aromatic dispenser 160. The control circuitry is also configured to control the aromatic dispenser to activate or deactivate it, adjust the rate aromatic aerosol is produced, adjust the type of aromatic dispensed, or other functionality including the functionality discussed above with regard to the air purifying device 102.

In some examples, this aerosol dispenser 160 is coupled to the air purifying device 102 to dispense an aromatic that is combined with the condensate and aerosolized by the aerosol generator. In some examples, the aerosol dispenser may be a separate device and include its own aerosol generator for generating an aromatic aerosol.

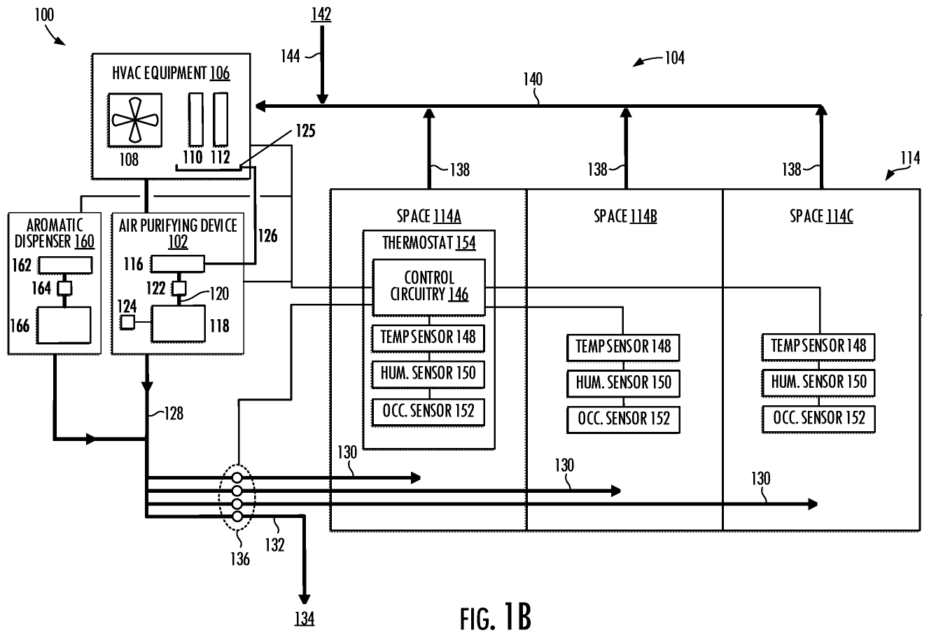

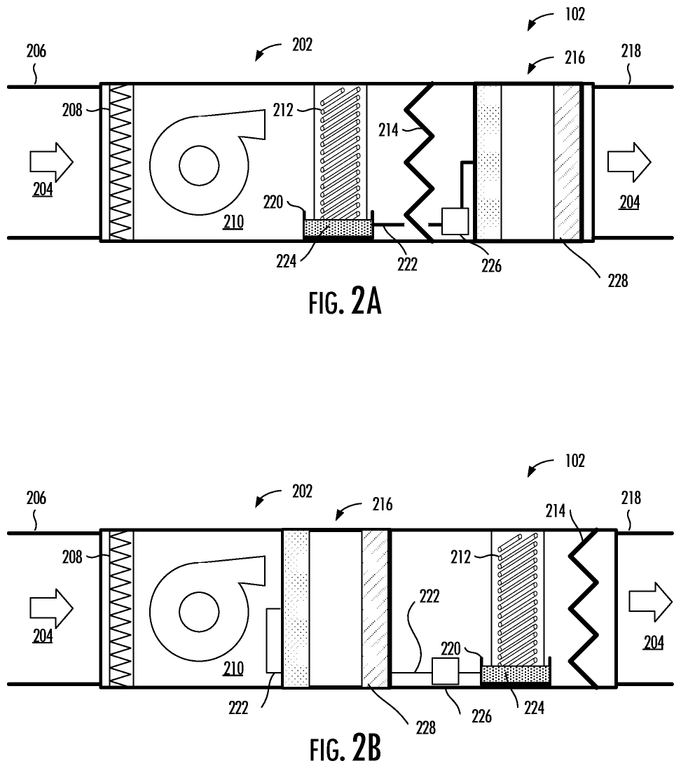

FIG. 2A shows a schematic of the components that may comprise the air purifying device 102 along with components of the HVAC system 104. In this example, these components are located within an air handling unit 202 and the conditioned air path is indicated by arrow 204. Return air from a conditioned space and/or outdoor air are directed to the air handling unit via return air duct 206. In this example, the air first passes through a mechanical filter 208, before entering an inlet of the fan 210. The fan pressurizes the air and directs it to the cooling element 212, and in this example, through a heating element 214 as well. The conditioned air is then directed through the aerosol generator 216, and finally into the supply air duct 218, which directs the air into a conditioned space (not shown).

In other implementations, the air handling unit 202 may be located vertically with cooling element 212 and/or the heating element 214 disposed above the fan 210. In other examples, the fan is located down stream of either the cooling element or the heating element. In still other examples, the cooling element and/or the heating element may be disposed within a fluid duct of the air handling unit and may also be selectively removable from the air handling unit. It is appreciated that more or less of these components may be utilized, and that they may be arranged in different configurations provided they provide the functionality described herein.

Condensate trap 220 collects condensate 224 and is shown coupled to the cooling element 212 in the depicted example in FIG. 2A. A conduit 222 couples the condensate trap to the aerosol generator 216, allowing the condensate created by the HVAC system 104 to be supplied to the aerosol generator. It is appreciated that the condensate trap may be associated with any condensate producing components within the overall device, including but not limited to, the heating element 214, the cooling element 212, or other components. In the depicted example, a condensate pump 226 is also used to move the condensate between the condensate trap and the aerosol generator.

While the condensate trap 220 is depicted as a U-shaped trap in this example, the condensate trap may comprise any suitable type of trap in other examples (e.g., P-shaped or S-shaped). The condensate trap may be configured to receive condensate from a drain pain 125 of an air handler, as illustrated schematically in FIG. 1A. For example, the drain pain may generally be arranged such that condensation may flow away from the drain pan and into the pipe 126 through the force of gravity, where it is directed into a condensate trap. In some examples, the condensate trap is the drain pan.

As shown in the depicted embodiment in FIG. 2A, the various components are located within an air handling unit 202. This configuration has various advantages, particularly due to its compact size. In addition, the components of the air purifying device 102 may be incorporated within an existing supply air path, potentially a supply air duct 218. In some examples, the air purifying device is associated with a ductless HVAC system 104 and located either within the ductless unit itself, coupled to the unit, or coupled to the conditioned space. In these examples, some or all of the various components within the air purifying device may be located within a plenum or other air channel directing the supply air out of the device. In other examples, one or more of these components may be located outside the supply air duct, or even outside the HVAC system.

FIG. 2B shows an alternative configuration with the same components. In this example, the aerosol generator 216 is located upstream of the cooling element 212 and the heating element 214. This configuration allows the aerosol generator to provide aerosol over these components, and in some examples, the aerosol generator cleans and/or disinfects the cooling element and/or the heating element. Other configurations may also be arranged and utilized.

In some examples, the components described herein may be included as a kit and may be added to an existing HVAC system 104. This kit may include some or all of the following components: the condensate trap 220, the conduit 222, the condensate pump 224, and the aerosol generator 216, all of which may be the same or similar to the components described herein. In some examples, these components also include brackets or extendable features allowing them to couple to and/or fit within the existing HVAC system.

FIG. 3 shows an example illustration of an aerosol generator 216 that may be used. The aerosol generator 216 may use various techniques to create an aerosol including negative ions from the condensate. In some examples, the aerosol generator uses water shearing, potentially the Lenard Effect, to generate the aerosol and the negative ions. In some examples, a field charger may create an electrical charge for ionizing the aerosol. This process at times is referred to as a corona effect. Other processes may be used for generating the aerosol and the negative ions, and these various processes may be used together in some instances.

The aerosol generator 216 shown in FIG. 3 uses both water shearing and a field charger. In the depicted example, the aerosol generator includes a bulk charging unit 302 and an electric field module 304. The condensate 224 may collect within the bulk charging unit to be generated into an aerosol 308. The bulk charging unit in this example further includes two piezo transducers 310 coupled to a drive circuit 312, which may be used to generate an aerosol by vibrating the condensate. In this example, the piezo transducers are positioned to engage with the condensate, and the drive circuit drives the piezo transducers, causing them to vibrate. This vibration shears and breaks up the condensate, which generates aerosol, typically in the form of water droplets 314, potentially liquid nanoparticles, and negative ions 316, which may be in the form of ionized water droplets or vapor. In some examples, these droplets form negative ions either directly from the energy provide via the transducers or through collisions with each other. In some examples, sprayers, or tortuous passages that shear the fluid, create an aerosol and negative ions.

In some examples, the aerosol generator 216 includes a mesh (not shown) coupled to piezo material and/or transducers. In these examples, the vibration of the piezo material causes the mesh to vibrate, which breaks up the condensate, creating the aerosol and negative ions. Other features or configurations may be used to vibrate and shear the condensate to create the aerosol and negative ions.

The drive circuit 312 is configured to drive the piezo transducer(s) 310, or in some examples a mesh, in a variety of different ways. The drive circuit may drive the piezo transducers or materials to generate ultrasonic waves within the condensate. These ultrasonic waves may vary in frequency and intensity, which may impact the aerosol generated. In some examples, the drive circuit drives the pizeo at a power level and frequency to generate very small particles such as liquid microparticles or even the smaller liquid nanoparticles. In these examples, the drive circuit may generate 70% or more liquid nanoparticles. In some of these examples, the amount and/or percentage of nanoparticles generated varies. In some examples, the drive circuit generates microparticles, which may be generated with the nanoparticles.

In some examples, were the system includes multiple piezo transducers or materials, the drive circuit 312 energizes the piezo transducers 310 or material to synchronously vibrate. This may include energizing these piezo transducers and/or material at the same frequency and power level. In other examples, the drive circuit energizes the piezo transducers or material to asynchronously vibrate. In these examples, this may include energizing these piezo transducers and/or materials at the different frequencies and/or power level. This asynchronous vibration may direct vibration energy through the condensate in a given direction or pattern, facilitating different amounts of aerosol generation, different sized droplets, and/or additional ionized aerosol particles.

In some examples, the aerosol generator 216 further includes an electric field module 304 arranged downstream from the bulk charging electrolytic unit 302. The electric field module may provide an electrical charge to the aerosol generated by the bulk charging electrolytic unit. In some examples, the electric field module may include a positive electrode 318 and a negative electrode 320. When the electric field module is energized, these electrodes energize the aerosol 308 and surrounding space. In some examples, this electric field charges the water aerosol droplets 314, creating more negative ions 316. In some examples, the aerosol generator is configured as an enclosed system, in which case aerosol generated by the system is comprised of only (or substantially only) aerosol generated from the condensate, which allows the field charger to energize primarily (or exclusively) the aerosol generated from the condensate. In some examples, the electric field module is a field charger and uses the corona effect to generate the electric field and in turn the negative ions.

In some examples, the aerosol generator 216 further includes a filter 260 as shown in FIG. 2A. This filter may be positioned downstream from the bulk charging electrolytic unit 302 and an electric field module 304. In some examples, the filter engages with the aerosol and negative ions after they have combined with the conditioned air. This may allow the aerosol and negative ions to interact with any pollutants that may be present in the conditioned air prior to passing through the filter. In some instances, the filter includes a charge, potentially a positive charge, which attracts any pollutants that have been negatively charged by the ions generated within the aerosol generator. In these instances, the filter may remove these pollutants from the air stream. In some examples, the filter is sized to remove any larger aerosol particles which may be undesirable in the conditioned air supplied to the spaces.

FIG. 4 shows an example of a condensate trap 220 that may be used in some implementations of the present air purifying device 202. In the example depicted in FIG. 2A, the condensate trap is coupled to the cooling element 212, however, as discussed above, the condensate trap may be coupled to any condensate generating component. In addition, the condensate trap may include additional features to filter and disinfect the condensate 306, including a particle filter 402 and an ultraviolet light source 404.

The particle filter 402 may be one or more filters designed to remove solid particles from the condensate 306. These filters may be removable filters, and potentially disposable. In some examples multiples filters are arranged to clean and purify the water collected within the condensate trap.

The ultraviolet light source 404 may comprise a plurality of lights energized to disinfect the condensate. In the example depicted in FIG. 4, a single light is submerged within the condensate 306. However, multiple lights may be used and arranged in a different matter, such as over the condensate, coupled to the particle filter, etc. In addition, the particle filter 402 and/or ultraviolet light source may be coupled to the condensate trap 220 discharge and/or conduit 222. In some examples, the condensate trap includes an anti-microbial material that prevents mold or other contaminants to grow on the condensate trap. Some examples only include one of these components, and other examples include none at all.

In some examples, the condensate trap 220 includes an outlet coupled to the conduit 222 for routing the condensate 306 to the aerosol generator 216. The conduit 222 may be coupled to a condensate pump 406 that pressurizes the condensate, moving it from the condensate trap to the aerosol generator. The condensate trap may further include at least one additional outlet connected to a discharge drain 408. The discharge drain may be utilized when the aerosol generator is not in use and/or when the condensate collected by the condensate trap exceeds the demands of the aerosol generator. In some examples, the discharge drain may be coupled to the condensate trap above the outlet for the conduit, which may allow the discharge drain to serve as the overflow drain for the condensate trap.

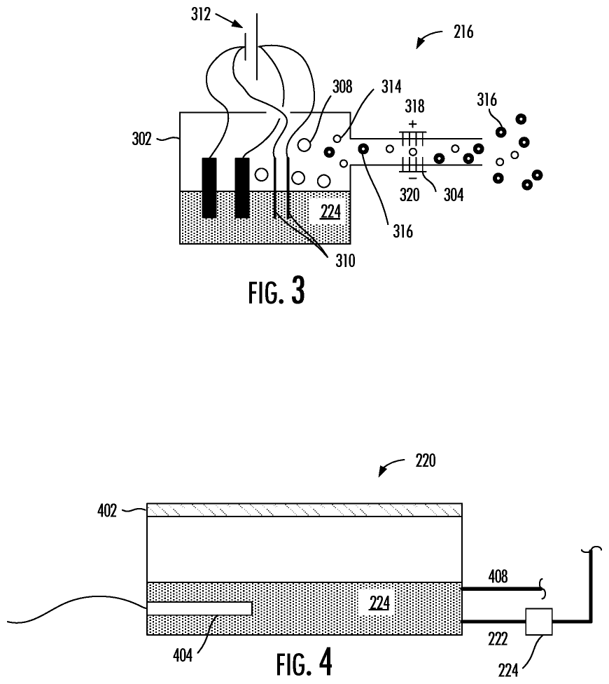

FIGS. 5A and 5B show an example illustration of an aromatic dispenser 160 that may be used in some implementations of the present air purifying device 202. The aromatic dispenser may be configured to supply an aromatic 502 that can be combined with the conditioned air provided by the HVAC system 104. The combination of the aromatic and the conditioned air may be delivered to a conditioned space, and in some examples, the aromatic and the conditioned air are combined before they reach the conditioned space. The aromatic dispenser include various features discussed above or may be a separate device. In some examples, the aromatic dispenser is incorporated within various features discussed above, for example, the aromatic dispenser may be included part of the aerosol generator 216.

FIG. 5A shows an example illustration of the aromatic dispenser 160, which may include some or all of the following components: an aromatic 502, a dispenser 504, a fluid 506, a fluid storage tank 508, a fluid supply 510, an aromatic conduit 512, and an aromatic aerosol generator 514 for generating an aromatic aerosol. Each of these components and how they interrelate are discussed more below.

The example shown in FIG. 5A is illustrative of how various components of the aromatic dispenser 160 may interact. In this example, the aromatic 502 is supplied into the fluid 506 using a dispenser 504. This fluid is contained within the fluid storage tank 508, and in this example, the aromatic and the fluid combine within the fluid storage tank to create an aromatic fluid. The aromatic fluid may then be routed via the aromatic conduit 512 to an aromatic aerosol generator 514 configured to energize the aromatic to generate an aromatic aerosol. The aromatic aerosol may be combined with the conditioned air and supplied to a conditioned space.

FIG. 5B show example illustrations of the dispenser 504, and in this example, the aromatic dispenser 160 includes multiple dispensers. In the example shown each dispenser includes a different constituent aromatic, e.g., one dispenser includes aromatic 502a and the other includes aromatic 502b. Other examples may include multiple dispensers where one or more dispensers include the same constituent aromatic. In addition, while two dispensers are shown, it is understood that more or less dispensers may be used. It is also understood that this illustration is only an example configuration of dispensers that may be used to dispense and/or combine aromatics.

In the example shown in FIG. 5B, the dispenser includes a container 516 for housing the aromatic 502 and an injector 518 for supplying the aromatic into the fluid 506 (shown in FIG. 5A). The dispenser 504 may direct the aromatic 502 from the container via the injector into the fluid 506. In this example, the fluid is contained within the fluid storage tank 508 where the fluid and the aromatic may be combined. In some examples, the dispenser may supply the aromatic into the fluid supply 510 (e.g., a water line), which may feed into the fluid storage tank. In other examples, the dispenser may supply the aromatic into the discharge from the fluid storage tank. In some examples, a fluid storage tank may not be necessary.

In some examples, the aromatic dispenser 160 uses the dispenser 504 to supply the aromatic 502 into the condensate 224 to produce an aromatic condensate. In these examples, the fluid storage tank 508 may be the condensate trap 220, and the aromatic is combined with the condensate in the condensate trap. In some examples, the aromatic may be dispensed into a water supply line that feeds into the condensate trap, which allows the aromatic, the water, and the condensate all to be combined together. In some examples, the aromatic is dispensed into the condensate after the condensate has left the condensate trap.

In some examples, the aromatic dispenser 160 may combine multiple different aromatics 502. For example, the aromatic dispenser may supply two or more constituent aromatics (502a and 502b) that are individually combinable with the conditioned air. In these examples, the aromatic dispenser may include one or more dispensers 504 with a given aromatic (e.g., lemon scented aromatic) and one or more other dispensers with another, different aromatic (e.g., lilac scented aromatic). The system may further control the various dispensers to only dispense a given constitute aromatic at a time, e.g., only lemon scent at a first time and only lilac scent at a second time.

In other examples, the aromatic dispenser 160 supply two or more constituent aromatics (502a and 502b) that are collectively combinable with the conditioned air. In these examples, the system may dispense two or more constitutes aromatics at the same time, e.g., combining lemon scented aromatic with the lilac scented aromatic, to create an aromatic that is different form either constitute aromatic on its own (e.g., a lemon-lilac scented aromatic). This may allow the aromatic dispenser to create multiple aromatics from a limited number of dispensers.

The control circuity 146 may control these dispensers 504 to allow the aromatic dispenser to supply different aromas for different purposes. For example, aromatic dispenser may supply aromatics based on a schedule. For example, one of the aromatics may be supplied during the daytime and another may be supplied at night. The aromatics (or lack thereof) may also correspond to various events such as a meal, a meeting, a cleaning event, an alarm event, a bedtime, etc. In some examples, the aromatic may be based on a user selection, potentially provided through the controller, and the user may make changes to the aromatic at any time. Various methods may be used for controlling these dispensers may also be used.

The control circuity 146 may also control the rate at which the aromatic is supplied. For example, the dispenser 504 may control the rate at which aromatic is supplied into the fluid. In some examples, this rate may be adjusted based on inputs to the system including various sensors, users input, or other system operations.

In some examples, the dispenser 504 includes a modulating valve 520 or variable opening for adjusting the supply, and/or rate of supply, of the aromatic 502 into the fluid 506. Any standard modulating valve (e.g., ball valve, butterfly valve, etc.) or standard technique for adjusting flow may be used. In addition, in some examples, the dispenser (and potentially the modulating valve or adjustable opening) is connected to the control circuity 146 and may be controlled based on various inputs such as system operating parameters, sensors, user input, etc. to adjust the flow or supply of aromatic form the dispenser(s).

The aromatic 502 used with the aromatic dispenser 160 may come in a variety of different forms. For example, a water-soluble liquid may be used and included within the container 516 of the dispenser 504. Other examples may use aromatics that are water soluble tablets, which may also be located within the container of the dispenser 504. In examples that use water soluble tablets, the injector 518 may be a lever or a sliding wall that moves the water soluble tablets from the container into the fluid. Other mechanisms may also be used.

In some examples, the aromatic 502 is directly dispensed by an individual, potentially a technician, into the fluid to be supplied to the aromatic aerosol generator 514. In these examples, the tablet aromatic, for example, may be dispensed into a water source or the condensate trap directly by an individual.

In some examples, the aromatic dispenser 160 may also include a fluid storage tank 508. This fluid storage tank may retain the fluid 506 to be combined with the aromatic 502. In some examples, the fluid storage tank may also serve to combine and/or mix the fluid with one or more constitute aromatics. In some examples, a fluid supply 510 is coupled to fluid storage tank to provide a consistent source of fluid. In some examples, the fluid is water and the fluid supply is a water line. In some examples, the fluid storage tank is the condensate trap, and in some of these examples, the fluid storage tank may not include a fluid supply.

In some examples, the aromatic dispenser 160 may also include an aromatic conduit 512 to supply the fluid combined with the aerosol to the aromatic aerosol generator 514. In some examples, the aromatic conduit is coupled to the fluid storage tank 508 and directs the fluid to the aromatic aerosol generator. In some examples, the aromatic conduit directs the aromatic directly from the dispenser 504 to the aromatic aerosol generator. In some examples, a pump similar to the pump 226 is necessary to move the fluid combined with the aerosol through the aromatic conduit. In some examples, the aromatic conduit is conduit 222, and in some examples the aromatic conduit is substantially the same as conduit 222.

In some examples, the aromatic aerosol generator 514 is the same or substantially similar to the aerosol generator 216 discussed above. The aromatic aerosol generator may include some or all of the components of aerosol generator 216. In some examples, the aromatic dispenser is coupled to the condensate trap 220. In these examples, the dispenser 504 may supply the aromatic to be combined with the condensate in the condensate trap. The condensate trap may serve as the fluid storage tank 508, and In these examples, the aerosol generator coupled to the condensate trap may serve as the aromatic aerosol generator for the aromatic dispenser. In other examples, the aromatic dispenser may be a separate system and may include a separate aromatic aerosol generator 514 for generating the aromatic aerosol.

In some examples, the aromatic aerosol generator 514 is controlled such that it only generates aromatic aerosol when the aerosol includes negative ions. In examples that include both an aerosol generator 216 and a separate aromatic aerosol generator 160, this control may include controlling the aromatic aerosol generator such that it only operates when the aerosol generator is operating. In embodiments, where the aerosol generator and the aromatic aerosol generator are the same device, this control may include controlling the dispenser 506 to only dispense aromatic 502 when the transducers 310 are vibrating at a frequency sufficient to produce negative ions.

Returning to FIGS. 1A and 1B, the HVAC system 104 may include an air circulation path comprising the supply air ducts 128, 130, 132 and the return air ducts 138, 140, 144, which directs the conditioned air. In some examples, the HVAC system may include only supply air ducts or only return air ducts, or no ducts at all. Regardless, the conditioned air in this path may be combined with the aerosol generated by the air purifying device 102, and in some examples, the aerosol includes negative ions. The air circulation path may direct this conditioned air, along with the aerosol, into one or more conditioned spaces 114. The air circulation path may further direct the conditioned air with the aerosol to targeted locations that may be particularly susceptible to air or surface contaminants. These spaces may include restrooms, dining rooms, meeting rooms, or in some cases specific locations within a room such as a water closet, work out space, or other locations. In some examples, as shown in FIG. 1, the air circulation path may direct conditioned air with the aerosol to an outdoor environment 134, potentially the ingress or egress locations for a building or conditioned space. This air may also be directed to outdoor congregation locations, or even the outdoor environment generally.

In some examples, the aromatic dispenser 160 is coupled to the HVAC system 104 and supplies the aromatic aerosol into the air circulation path to be combined with the conditioned air. In some examples, the aromatic aerosol is supplied into a supply air duct. In some examples, the aromatic aerosol is supplied via the air purifying device.

In some examples, control circuitry 146 is coupled to the HVAC system 104, the air purifying device 102, and/or the aromatic dispenser 160. The control circuitry may be configured to control and monitor any of components discussed above. In some examples, the control circuitry is configured to control these components in the following manner.

As shown in FIGS. 1A and 1B, the control circuitry 146 may be operatively coupled to the HVAC system 104, the air purifying device 102, and/or the aromatic dispenser 160. In some examples, the control circuitry controls the air purifying device based on the occupancy of a given space or zone. In these examples, the control circuitry receives an indication of the occupancy of a given space or zone from one or more occupancy sensors 152. The control circuitry then controls the generation of the aerosol based on the occupancy measurements received. In these examples, the control circuitry may seek to initiate or increase the quantity of aerosol to a space based on the occupancy of the space. For example, the control circuitry may increase the quantity of aerosol generated as the occupancy increases, or conversely decrease the quantity of aerosol as occupancy declines. In some examples, a minimum and/or maximum aerosol quantity is set for the system.

In some instances, the control circuitry 146 may control the air purifying device 102, and potentially HVAC equipment 106, based on the occupancy measurements for a given space independent of any cooling or heating load. For example, in some instances, the occupancy sensor 152 may indicate a space is occupied, however, the temperature sensor 148 or other conditioning sensors indicates that the space is within acceptable temperature parameters. In these instances, the control circuitry may initiate an air cleaning mode, whereby the air purifying device generates aerosol and negative ions, and the fan 108 circulates air combined with the aerosol and negative ions to the space without conditioning the air, e.g., without activating the heating or the cooling element. In some of these examples, a separate water supply line (not shown) may be coupled to the condensate trap in the event insufficient condensate is available.

In some examples, the control circuitry 146 receives scheduling information for one or more conditioned space(s) or zone(s). This information may be in any form including an estimate of occupancy, heating/cooling load, or otherwise, for a given space/zone at a given time. In some examples, this scheduling information is arranged in time intervals. In these examples, the control circuitry may control the aerosol generator based on, at least in part, the expected load for at a given time interval.

For example, the expected conditioning load for one-time interval, potentially a first time interval, may be less than expected conditioning load for another time interval, potentially a second time interval. This change in load may indicate that the space will be occupied during the second time interval. In these instances, the control circuitry may increase the quantity of aerosol generated during the first time interval in advance of the second time interval. In some instances, this may include initiating an air cleaning mode if the space or zone does not have any heating or cooling load at that time. This may allow for the system to clean and disinfect the conditioned space in advance of a given event, e.g., a meeting, etc.

In some instances, the production of aerosol is continued for some or all of the event. In some examples, the aerosol generation and distribution is initiated near or at the end of the event or time interval. In some examples, the aerosol is generated and directed into the space for a short period of time, such as 5 minutes, 10 minutes, or 15 minutes. In other examples, the time period is longer, one hour or more. In some examples, the aerosol is generated and directed into the space until the air within the room is partially or fully replaced.

The control circuitry may also control the aromatic dispenser in a similar manner in response to occupancy measurements, and in some examples, the air purifying device and the aromatic dispenser are controlled jointly in response to occupancy measurement.

In some examples, the control circuitry 146 includes humidity sensors 150 and controls the air purifying device 102 and/or HVAC system 104 based on humidity information received from the humidity sensors. The humidity sensors in these examples may be positioned within a space to measure the humidity level generated by conditions of the space, including any outdoor air provided to the system along with the humidity generated by the air purifying device. The control circuitry may control the HVAC system based on these humidity measurements.

For example, the control circuitry 146 may ensure the humidity levels within a space are within a given humidity range. If the humidity measured in the space is over an acceptable or desired humidity level, the control circuitry may seek to dehumidify the conditioned air supplied to the space. In some of these examples, this may involve increasing the cooling relief applied to the conditioned air by the cooling element 110. If this increase in cooling relief impacts the temperature of the space in an undesirable manner, the heating element 112 may be used, potentially in a reheat mode. In some examples, the control circuitry decreases or shuts off the amount of aerosol generated 118 by the air purifying device in response to high humidity measurements. In instances where the humidity level is measured to be below a desired or acceptable humidity range, the control circuitry may initiate or increase the volume of aerosol generated by the air purifying device.

In some examples, the control circuitry 146 may adjust a rate at which aerosol is generated by the air purifying device 102 based on the measurements of the humidity level. In these examples, the control circuitry may decrease the rate at which the aerosol is generated when the system receives an indication that a high humidity level is detected. For example, a humidity sensor in a conditioned room may detect a higher level of humidity than desired, or the humidity level may be above a given threshold or range. In response to this humidity measurement, the control circuitry may control the air purifying device to decrease the aerosol being produced. The control circuity may also operate the system conversely to increase the supply of aerosol provided by the air purifying device in response to a low level of humidity being detected, e.g., a level of humidity below a threshold level, range or a desired level.

In some examples, the control circuitry 146 is configured to adjust a particle size of the aerosol generated by the air purifying device 142 based on the measurements of the humidity level. In these examples, the control circuitry may decrease the particle size of the aerosol generated by the air purifying device in response to the system receiving an indication that a high humidity level is detected. For example, a humidity sensor in a conditioned room may detect a higher level of humidity than desired, or the humidity level may be above a given threshold or range. In response to this humidity measurement, the control circuitry may control the air purifying device to decrease particle size of the aerosol being produced. For example, the air purifying device may be controlled to adjust the frequency or power provided to the transducers to reduce the size of the particles produced and/or increase the percentage of smaller particles (e.g., microparticles or nanoparticles) produced. The control circuity may also operate the system conversely to increase the particle size of the aerosol provided by the air purifying device in response to a low level of humidity being detected, e.g., a level of humidity below a threshold level, range or a desired level.

The control circuitry may also control the aromatic dispenser in a similar manner in response to humidity measurements, and in some examples, the air purifying device and the aromatic dispenser are controlled jointly in response to humidity measurement.

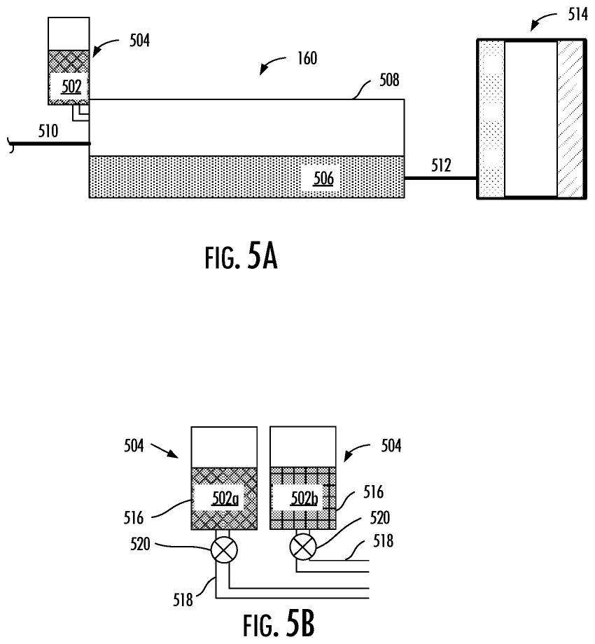

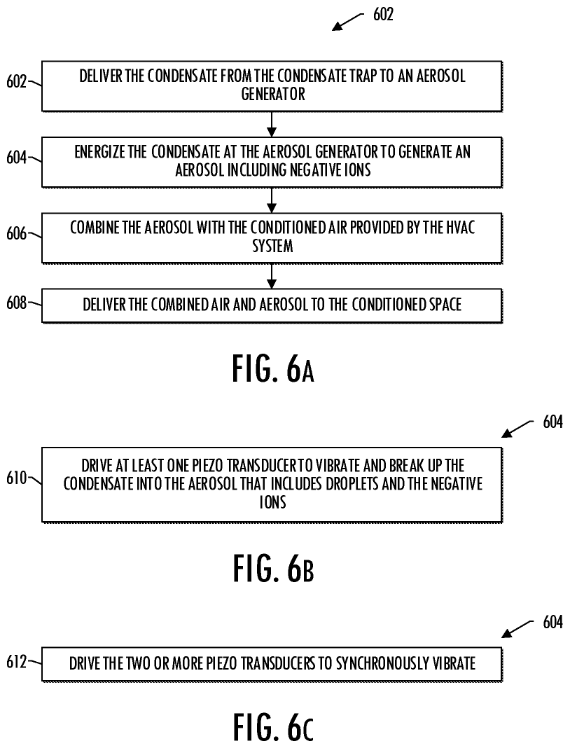

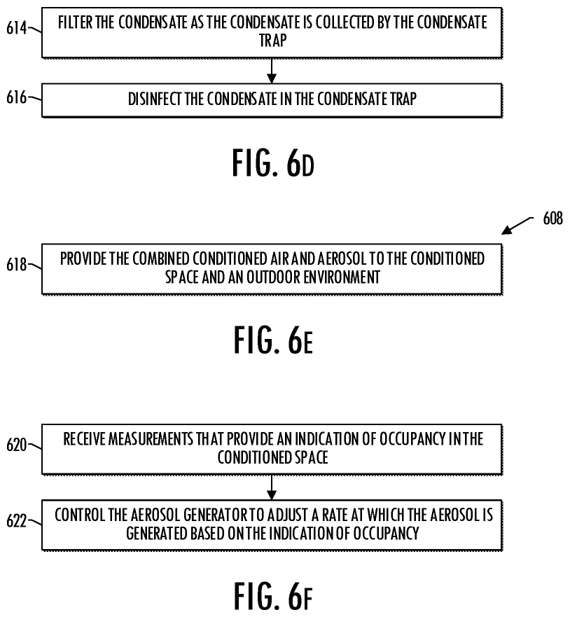

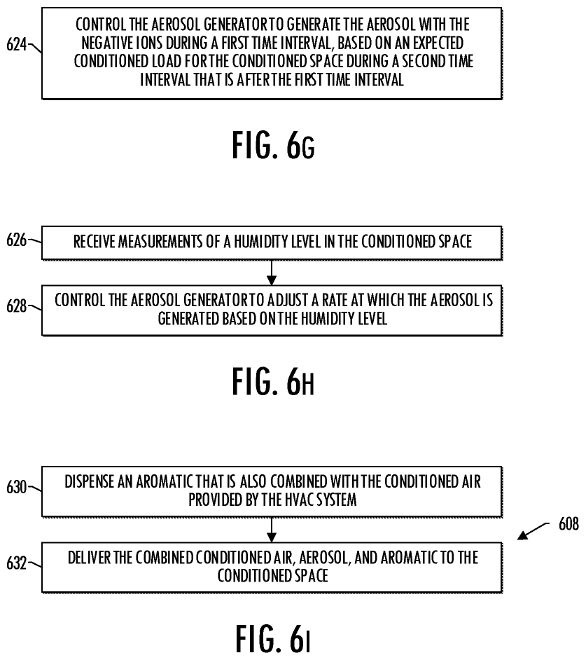

FIGS. 6A-6I are flowcharts illustrating various steps in a method 600 of disinfecting a conditioned space to which heating ventilation, and air conditioning (HVAC) equipment 106 of an HVAC system 104 is configured to provide conditioned air, the HVAC system including a condensate trap 220 configured to collect condensate 224 produced during operation, according to various example implementations of the present disclosure. The method includes delivering the condensate from the condensate trap to an aerosol generator 216, as shown at block 602 of FIG. 6A. The method includes energizing the condensate at the aerosol generator to generate an aerosol including negative ions, as shown at block 604. The method includes combining the aerosol with the conditioned air provided by the HVAC system, as shown at block 606. And the method includes delivering the combined air and aerosol to the conditioned space, as shown at block 608.

In some examples, energizing the condensate at block 604 includes driving at least one piezo transducer to vibrate and break up the condensate into the aerosol that includes droplets and the negative ions, as shown at block 610 of FIG. 6B.

In some examples, the aerosol is generated such that the droplets are liquid nanoparticle droplets. In some examples, the aerosol is generated such that the droplets are liquid microparticles.

In some examples, the at least one piezo transducer is two or more piezo transducers, and energizing the condensate at block 604 further includes driving the two or more piezo transducers to synchronously vibrate, as shown at block 612 of FIG. 6C.

In some examples, the method 600 further includes filtering the condensate as the condensate is collected by the condensate trap, and disinfecting the condensate in the condensate trap, as shown at blocks 616 and 614 of FIG. 6D.

In some examples, the delivering the combined air and aerosol at block 608 further includes providing the combined conditioned air and aerosol to the conditioned space and an outdoor environment, as shown at block 618 of FIG. 6E.

In some examples, the method 600 further includes receiving measurements that provide an indication of occupancy in the conditioned space, as shown at block 620 of FIG. 6F. And the method includes controlling the aerosol generator to adjust a rate at which the aerosol is generated based on the indication of occupancy, as shown at block 622.

In some examples, the method 600 further includes controlling the aerosol generator to generate the aerosol with the negative ions during a first time interval, based on an expected conditioned load for the conditioned space during a second time interval that is after the first time interval, as shown at block 624 of FIG. 6G.

In some examples, the method 600 further includes receiving (626) measurements of a humidity level in the conditioned space, as shown at block 626 of FIG. 6H. And the method includes controlling the aerosol generator to adjust a rate at which the aerosol is generated based on the humidity level, as shown at block 628.

In some examples, the method 600 further includes dispensing an aromatic that is also combined with the conditioned air provided by the HVAC system, as shown at block 630 of FIG. 6I. In some of these examples, delivering the combined air and aerosol at block 608 includes delivering the combined conditioned air, aerosol, and aromatic to the conditioned space, as shown at block 632.

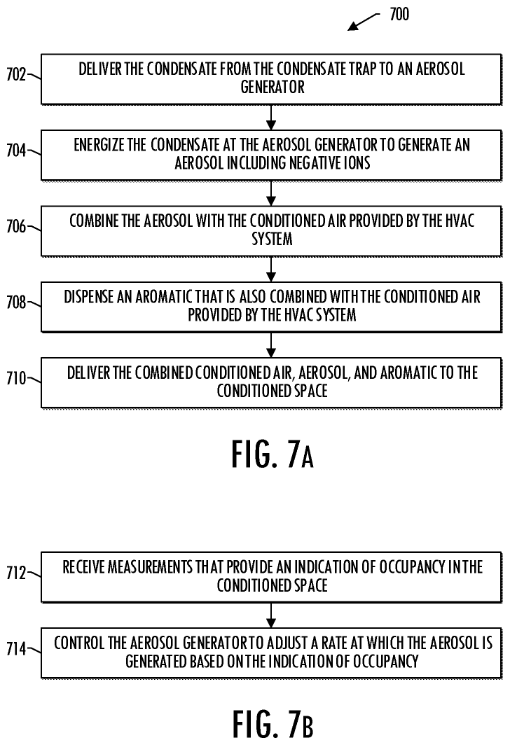

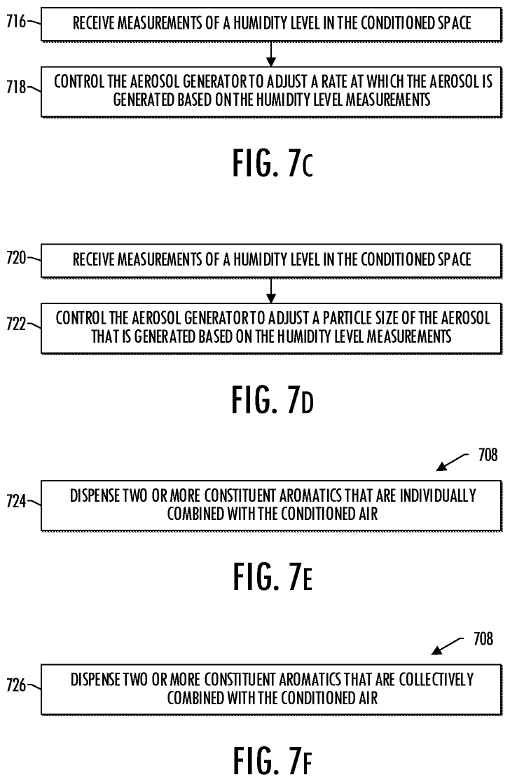

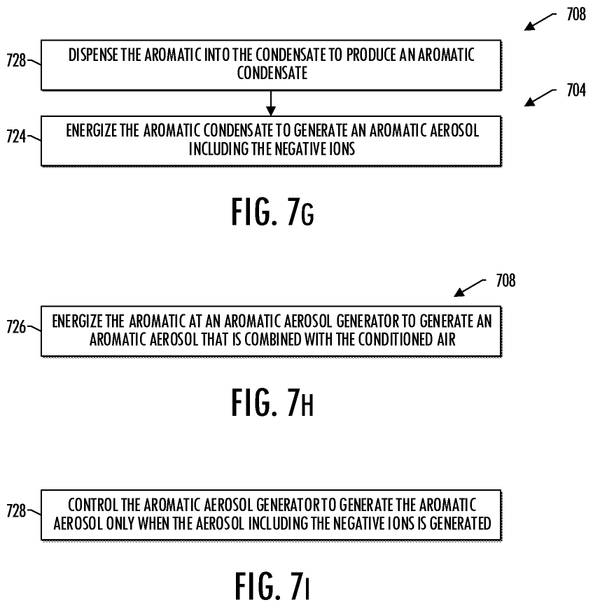

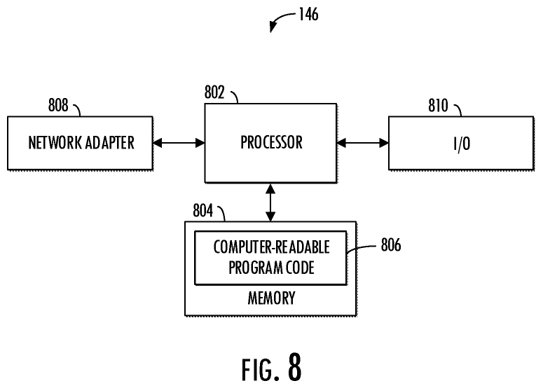

FIGS. 7A-7I are flowcharts illustrating various steps in a method 700 of disinfecting a conditioned space to which heating ventilation, and air conditioning (HVAC) equipment 106 of an HVAC system 104 is configured to provide conditioned air, the HVAC system including a condensate trap220 configured to collect condensate 224 produced during operation, according to various example implementations of the present disclosure. The method includes delivering the condensate from the condensate trap to an aerosol generator, as shown at block 702 of FIG. 7A. The method includes energizing the condensate at the aerosol generator to generate an aerosol including negative ions, as shown at block 704. The method includes combining the aerosol with the conditioned air provided by the HVAC system, as shown at block 706. The method includes dispensing an aromatic 502 that is also combined with the conditioned air provided by the HVAC system, as shown at block 708. And the method includes delivering the combined conditioned air, aerosol, and aromatic to the conditioned space, as shown at block 710.

In some examples, the method 700 further includes receiving measurements that provide an indication of occupancy in the conditioned space, as shown at block 712 of FIG. 7B. In some of these examples, the method also includes controlling the aerosol generator to adjust a rate at which the aerosol is generated based on the indication of occupancy, as shown at block 714.

In some examples, the method 700 further includes receiving measurements of a humidity level in the conditioned space, as shown at block 716 of FIG. 7C. In some of these examples, the method also includes controlling the aerosol generator to adjust a rate at which the aerosol is generated based on the humidity level measurements, as shown at block 718.

In some examples, the method 700 further includes receiving measurements of a humidity level in the conditioned space, as shown at block 720 of FIG. 7D. In some of these examples, the method also includes controlling the aerosol generator to adjust a particle size of the aerosol that is generated based on the humidity level measurements, as shown at block 722.

In some examples, dispensing the aromatic at block 708 includes dispensing two or more constituent aromatics that are individually combined with the conditioned air, as shown at block 724 of FIG. 7E.

In some examples, dispensing the aromatic at block 708 includes dispensing two or more constituent aromatics that are collectively combined with the conditioned air, as shown at block 726 of FIG. 7F.

In some examples, the aromatic is a water soluble tablet.

In some examples, the dispensing the aromatic at block 708 includes dispensing the aromatic into the condensate to produce an aromatic condensate, as shown at block 728 of FIG. 7G. In some of these examples, energizing the condensate at block 704 includes energizing the aromatic condensate to generate an aromatic aerosol including the negative ions, as shown at block 724.

In some examples, the dispensing the aromatic at block 708 further includes energizing the aromatic at an aromatic aerosol generator to generate an aromatic aerosol that is combined with the conditioned air, as shown at block 726 of FIG. 711.

In some examples, the method 700 further includes controlling the aromatic aerosol generator to generate the aromatic aerosol only when the aerosol including the negative ions is generated, as shown at block 728 of FIG. 7I.

FIG. 8 illustrates the control circuitry 146 according to some example implementations of the present disclosure. The control circuitry may include one or more of each of a number of components such as, for example, a processor 802 connected to a memory 804. The processor is generally any piece of computer hardware capable of processing information such as, for example, data, computer programs and/or other suitable electronic information. The processor includes one or more electronic circuits some of which may be packaged as an integrated circuit or multiple interconnected integrated circuits (an integrated circuit is at times more commonly referred to as a “chip”). The processor 802 may be a number of processors, a multi-core processor, or some other type of processor, depending on the particular implementation.

The processor 802 may be configured to execute computer programs such as computer-readable program code 806, which may be stored onboard the processor or otherwise stored in the memory 804. In some examples, the processor may be embodied as or otherwise include one or more ASICs, FPGAs or the like. Thus, although the processor may be capable of executing a computer program to perform one or more functions, the processor of various examples may be capable of performing one or more functions without the aid of a computer program.

The memory 804 is generally any piece of computer hardware capable of storing information, such as, for example, data, computer-readable program code 606 or other computer programs, and/or other suitable information either on a temporary basis and/or a permanent basis. The memory may include volatile memory such as random access memory (RAM), and/or non-volatile memory such as a hard drive, flash memory or the like. In various instances, the memory may be referred to as a computer-readable storage medium, which is a non-transitory device capable of storing information. In some examples, then, the computer-readable storage medium is non-transitory and has computer-readable program code stored therein, which, in response to execution by the processor 802, causes the control circuitry 146 to perform various operations as described herein, some of which may in turn cause the air purifying system 100 to perform various operations.

In addition to the memory 804, the processor 802 may also be connected to one or more peripherals such as a network adapter 808, one or more input/output (I/O) devices 810, or the like. The network adapter is a hardware component configured to connect the control circuitry 146 to a computer network to enable the control circuitry to transmit and/or receive information via the computer network. The I/O devices may include one or more input devices capable of receiving data or instructions for the control circuitry, and/or one or more output devices capable of providing an output from the control circuitry. Examples of suitable input devices include a keyboard, keypad or the like, and examples of suitable output devices include a display device such as a one or more light-emitting diodes (LEDs), a LED display, a liquid crystal display (LCD), or the like,

As explained above and reiterated below, the present disclosure includes, without limitation, the following example implementations.

Clause 1. An air purifying system comprising: a heating, ventilation and air conditioning (HVAC) system with HVAC equipment configured to provide conditioned air to a conditioned space, the HVAC system including a condensate trap configured to collect condensate produced during operation; and an air purifying device configured to energize the condensate from the condensate trap to generate an aerosol including negative ions, the aerosol being combinable with the conditioned air provided by the HVAC system for delivery to the conditioned space.

Clause 2. The air purifying system of clause 1, wherein the HVAC system is set up in an installation that defines an air supply path, and wherein the air purifying device includes an aerosol generator located in the air supply path and configured to energize the condensate.

Clause 3. The air purifying system of clause 2, wherein the HVAC equipment includes a heat exchanger coil located in the air supply path, wherein the heat exchanger coil is arranged downstream from the aerosol generator in the air supply path.

Clause 4. The air purifying system of any of clauses 1 to 3, further comprising: a particle filter configured to filter the condensate as the condensate is collected by the condensate trap; and an ultraviolet light source configured to disinfect the condensate in the condensate trap.

Clause 5. The air purifying system of any of clauses 1 to 4, wherein the HVAC system is configured to provide the conditioned air combined with the aerosol including the negative air ions, to the conditioned space and an outdoor environment.

Clause 6. The air purifying system of any of clauses 1 to 5, wherein the HVAC system further includes control circuitry operably coupled to the HVAC equipment and the air purifying device, and wherein the HVAC equipment further includes a sensor configured to provide an indication of occupancy in the conditioned space, and the control circuitry is configured to control the air purifying device to adjust a rate at which the aerosol is generated by the air purifying device based on the indication of occupancy,

Clause 7. The air purifying system of any of clauses 1 to 6, wherein the HVAC system further includes control circuitry operably coupled to the HVAC equipment and the air purifying device, and the control circuitry is configured to control the air purifying device to generate the aerosol with the negative ions during a first time interval, based on an expected conditioned load for the conditioned space during a second time interval that is after the first time interval.

Clause 8. The air purifying system of any of clauses 1 to 7, wherein the HVAC system further includes control circuitry operably coupled to the HVAC equipment and the air purifying device, and wherein the HVAC equipment further includes a humidity sensor configured to provide measurements of a humidity level in the conditioned space, and the control circuitry is configured to adjust a rate at which aerosol is generated by the air purifying device based on the measurements of the humidity level.

Clause 9. The air purifying system of any of clauses 1 to 8, further comprising an aromatic dispenser configured to dispense an aromatic that is also combinable with the conditioned air provided by the HVAC system for delivery to the conditioned space, the aromatic combinable with the conditioned air before the conditioned air reaches the conditioned space.

Clause 10. An air purifying system comprising: a heating, ventilation and air conditioning (HVAC) system with HVAC equipment configured to provide conditioned air to a conditioned space, the HVAC system including a condensate trap configured to collect condensate produced during operation; an air purifying device configured to energize the condensate from the condensate trap to generate an aerosol including negative ions, the aerosol being combinable with the conditioned air provided by the HVAC system for delivery to the conditioned space; and an aromatic dispenser configured to dispense an aromatic that is also combinable with the conditioned air provided by the HVAC system for delivery to the conditioned space, the aromatic combinable with the conditioned air before the conditioned air reaches the conditioned space.

Clause 11. The air purifying system of clause 10, wherein the HVAC system is set up in an installation that defines an air supply path, and wherein the air purifying device includes an aerosol generator located in the air supply path and configured to energize the condensate.

Clause 12. The air purifying system of clause 10 or clause 11, wherein the HVAC equipment includes a sensor configured to provide an indication of occupancy in the conditioned space, and the HVAC system further includes control circuitry operably coupled to the HVAC equipment and the air purifying device, and wherein the control circuitry is configured to adjust a rate at which aerosol is generated by the air purifying device based on the indication of occupancy.

Clause 13. The air purifying system of any of clauses 10 to 12, wherein the HVAC equipment further includes a humidity sensor configured to provide measurements of a humidity level in the conditioned space, and the HVAC system further includes control circuitry operably coupled to the HVAC equipment and the air purifying device, and wherein the control circuitry is configured to adjust a rate at which aerosol is generated by the air purifying device based on the measurements of the humidity level.

Clause 14. The air purifying system of any of clauses 10 to 13, wherein the HVAC equipment further includes a humidity sensor configured to provide measurements of a humidity level in the conditioned space, and the HVAC system further includes control circuitry operably coupled to the HVAC equipment and the air purifying device, and wherein the control circuitry is configured to adjust a particle size of the aerosol that is generated by the air purifying device based on the measurements of the humidity level.

Clause 15. The air purifying system of any of clauses 10 to 14, wherein the aromatic dispenser is configured to dispense two or more constituent aromatics that are individually combinable with the conditioned air.

Clause 16. The air purifying system of any of clauses 10 to 15, wherein the aromatic dispenser is configured to dispense two or more constituent aromatics that are collectively combinable with the conditioned air.

Clause 17. The air purifying system of any of clauses 10 to 16, wherein the aromatic is a water soluble tablet.

Clause 18. The air purifying system of any of clauses 10 to 17, wherein the aromatic dispenser is configured to dispense the aromatic into the condensate using a dispenser to produce an aromatic condensate, and the air purifying device is configured to energize the aromatic condensate to generate an aromatic aerosol including the negative ions.

Clause 19. The air purifying system of any of clauses 10 to 18, wherein the aromatic dispenser includes an aromatic aerosol generator configured to energize the aromatic to generate an aromatic aerosol that is combinable with the conditioned air.

Clause 20. The air purifying system of clause 19, further comprising control circuitry configured to control the air purifying device to generate the aerosol including the negative ions, the control circuitry also configured to control the aromatic aerosol generator to generate the aromatic aerosol only when the aerosol including the negative ions is generated.

Clause 21. An air purifying device for a heating, ventilation and air conditioning (HVAC) system operable to provide conditioned air to a conditioned space. the HVAC system including a condensate trap configured to collect condensate produced during operation of the HVAC system, the air purifying device comprising: an aerosol generator; and a conduit operably coupleable to and between the condensate trap and the aerosol generator, the conduit configured to deliver the condensate from the condensate trap to the aerosol generator, the aerosol generator configured to energize the condensate to generate an aerosol including negative ions combinable with the conditioned air provided by the HVAC system for delivery to the conditioned space.

Clause 22. The air purifying device of clause 21, wherein the aerosol generator comprises: at least one piezo transducer; and a driver circuit configured to drive the at least one piezo transducer to vibrate and break up the condensate into the aerosol that includes droplets and the negative ions.

Clause 23. The air purifying device of clause 22, wherein the aerosol generator is configured such that the droplets are liquid nanoparticles.

Clause 24. The air purifying device of clause 22 or clause 23, wherein the at least one piezo transducer is two or more piezo transducers, and the driver circuit is configured to drive the two or more piezo transducers to synchronously vibrate.

Clause 25. The air purifying device of any of clauses 21 to 24, wherein the aerosol generator is configured to generate the aerosol with the negative ions under control from the HVAC system, and based on an indication of occupancy in the conditioned space.

Clause 26. The air purifying device of any of clauses 21 to 25, further comprising an aromatic dispenser configured to dispense an aromatic that is also combinable with the conditioned air provided by the HVAC system for delivery to the conditioned space, the aromatic combinable with the conditioned air before the conditioned air reaches the conditioned space.

Clause 27. A method of disinfecting a conditioned space to which heating, ventilation, and air conditioning (HVAC) equipment of an HVAC system is configured to provide conditioned air, the HVAC system including a condensate trap configured to collect condensate produced during operation, the method comprising: delivering the condensate from the condensate trap to an aerosol generator; energizing the condensate at the aerosol generator to generate an aerosol including negative ions; combining the aerosol with the conditioned air provided by the HVAC system; and delivering the combined air and aerosol to the conditioned space.

Clause 28. The method of clause 27, wherein energizing the condensate includes driving at least one piezo transducer to vibrate and break up the condensate into the aerosol that includes droplets and the negative ions.

Clause 29. The method of clause 28, wherein the aerosol is generated such that the droplets are liquid nanoparticle droplets.

Clause 30. The method of clause 28 or clause 29, wherein the aerosol is generated such that the droplets are liquid microparticles.

Clause 31. The method of any of clauses 28 to 30, wherein the at least one piezo transducer is two or more piezo transducers, and energizing the condensate further includes driving the two or more piezo transducers to synchronously vibrate.

Clause 32. The method of any of clauses 27 to 31, further comprising filtering the condensate as the condensate is collected by the condensate trap, and disinfecting the condensate in the condensate trap.

Clause 33. The method of any of clauses 27 to 32, wherein the delivering the combined air and aerosol further includes providing the combined conditioned air and aerosol to the conditioned space and an outdoor environment.

Clause 34. The method of any of clauses 27 to 33, further comprising: receiving measurements that provide an indication of occupancy in the conditioned space; and controlling the aerosol generator to adjust a rate at which the aerosol is generated based on the indication of occupancy.

Clause 35. The method of any of clauses 27 to 34, further comprising: controlling the aerosol generator to generate the aerosol with the negative ions during a first time interval, based on an expected conditioned load for the conditioned space during a second time interval that is after the first time interval.

Clause 36. The method of any of clauses 27 to 35, further comprising: receiving measurements of a humidity level in the conditioned space; and controlling the aerosol generator to adjust a rate at which the aerosol is generated based on the humidity level,

Clause 37. The method of any of clauses 27 to 36, further comprising dispensing an aromatic that is also combined with the conditioned air provided by the HVAC system, and wherein delivering the combined air and aerosol includes delivering the combined conditioned air, aerosol, and aromatic to the conditioned space.

Clause 38. A method of disinfecting a conditioned space to which heating, ventilation, and air conditioning (HVAC) equipment of an HVAC system is configured to provide conditioned air, the HVAC system including a condensate trap configured to collect condensate produced during operation, the method comprising: delivering the condensate from the condensate trap to an aerosol generator; energizing the condensate at the aerosol generator to generate an aerosol including negative ions; combining the aerosol with the conditioned air provided by the HVAC system; dispensing an aromatic that is also combined with the conditioned air provided by the HVAC system; and delivering the combined conditioned air, aerosol, and aromatic to the conditioned space.

Clause 39. The method of clause 38, further comprising: receiving measurements that provide an indication of occupancy in the conditioned space; and controlling the aerosol generator to adjust a rate at which the aerosol is generated based on the indication of occupancy.

Clause 40. The method of clause 38 or clause 39, further comprising: receiving measurements of a humidity level in the conditioned space; and controlling the aerosol generator to adjust a rate at which the aerosol is generated based on the humidity level measurements.

Clause 41. The method of any of clauses 38 to 40, further comprising: receiving measurements of a humidity level in the conditioned space; and controlling the aerosol generator to adjust a particle size of the aerosol that is generated based on the humidity level measurements.

Clause 42. The method of any of clauses 38 to 41, wherein dispensing the aromatic includes dispensing two or more constituent aromatics that are individually combined with the conditioned air.

Clause 43. The method of any of clauses 38 to 42, wherein dispensing the aromatic includes dispensing two or more constituent aromatics that are collectively combined with the conditioned air.

Clause 44. The method of any of clauses 38 to 43, wherein the aromatic is a water soluble tablet.

Clause 45. The method of any of clauses 38 to 44, wherein the dispensing the aromatic includes dispensing the aromatic into the condensate to produce an aromatic condensate, and energizing the condensate includes energizing the aromatic condensate to generate an aromatic aerosol including the negative ions.

Clause 46. The method of any of clauses 38 to 45, wherein the dispensing the aromatic further includes energizing the aromatic at an aromatic aerosol generator to generate an aromatic aerosol that is combined with the conditioned air.

Clause 47. The method of clause 46, further comprising controlling the aromatic aerosol generator to generate the aromatic aerosol only when the aerosol including the negative ions is generated.

Many modifications and other implementations of the disclosure set forth herein will come to mind to one skilled in the art to which the disclosure pertains having the benefit of the teachings presented in the foregoing description and the associated figures. Therefore, it is to be understood that the disclosure is not to be limited to the specific implementations disclosed and that modifications and other implementations are intended to be included within the scope of the appended claims. Moreover, although the foregoing description and the associated figures describe example implementations in the context of certain example combinations of elements and/or functions, it should be appreciated that different combinations of elements and/or functions may be provided by alternative implementations without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

What is claimed is:

1. An air purifying system comprising:
a heating, ventilation and air conditioning (HVAC) system with HVAC equipment configured to provide conditioned air to a conditioned space, the HVAC system including a condensate trap configured to collect condensate produced during operation;
an air purifying device configured to energize the condensate from the condensate trap to generate an aerosol including negative ions, the aerosol being combinable with the conditioned air provided by the HVAC system for delivery to the conditioned space; and
an aromatic dispenser configured to dispense an aromatic that is also combinable with the conditioned air provided by the HVAC system for delivery to the conditioned space, the aromatic combinable with the conditioned air before the conditioned air reaches the conditioned space.

2. The air purifying system of claim 1, wherein the HVAC system is set up in an installation that defines an air supply path, and
wherein the air purifying device includes an aerosol generator located in the air supply path and configured to energize the condensate.

3. The air purifying system of claim 1, wherein the HVAC equipment includes a sensor configured to provide an indication of occupancy in the conditioned space, and the HVAC system further includes control circuitry operably coupled to the HVAC equipment and the air purifying device, and
wherein the control circuitry is configured to adjust a rate at which aerosol is generated by the air purifying device based on the indication of occupancy.

4. The air purifying system of claim 1, wherein the HVAC equipment further includes a humidity sensor configured to provide measurements of a humidity level in the conditioned space, and the HVAC system further includes control circuitry operably coupled to the HVAC equipment and the air purifying device, and
wherein the control circuitry is configured to adjust a rate at which aerosol is generated by the air purifying device based on the measurements of the humidity level.

5. The air purifying system of claim 1, wherein the HVAC equipment further includes a humidity sensor configured to provide measurements of a humidity level in the conditioned space, and the HVAC system further includes control circuitry operably coupled to the HVAC equipment and the air purifying device, and
wherein the control circuitry is configured to adjust a particle size of the aerosol that is generated by the air purifying device based on the measurements of the humidity level.

6. The air purifying system of claim 1, wherein the aromatic dispenser is configured to dispense two or more constituent aromatics that are individually combinable with the conditioned air.

7. The air purifying system of claim 1, wherein the aromatic dispenser is configured to dispense two or more constituent aromatics that are collectively combinable with the conditioned air.

8. The air purifying system of claim 1, wherein the aromatic is a water soluble tablet.

9. The air purifying system of claim 1, wherein the aromatic dispenser is configured to dispense the aromatic into the condensate using a dispenser to produce an aromatic condensate, and the air purifying device is configured to energize the aromatic condensate to generate an aromatic aerosol including the negative ions.

10. The air purifying system of claim 1, wherein the aromatic dispenser includes an aromatic aerosol generator configured to energize the aromatic to generate an aromatic aerosol that is combinable with the conditioned air.

11. The air purifying system of claim 10, further comprising control circuitry configured to control the air purifying device to generate the aerosol including the negative ions, the control circuitry also configured to control the aromatic aerosol generator to generate the aromatic aerosol only when the aerosol including the negative ions is generated.

12. A method of disinfecting a conditioned space to which heating, ventilation, and air conditioning (HVAC) equipment of an HVAC system is configured to provide conditioned air, the HVAC system including a condensate trap configured to collect condensate produced during operation, the method comprising:
delivering the condensate from the condensate trap to an aerosol generator;
energizing the condensate at the aerosol generator to generate an aerosol including negative ions;
combining the aerosol with the conditioned air provided by the HVAC system;
dispensing an aromatic that is also combined with the conditioned air provided by the HVAC system; and
delivering the combined conditioned air, aerosol, and aromatic to the conditioned space.

13. The method of claim 12, further comprising:
receiving measurements that provide an indication of occupancy in the conditioned space; and
controlling the aerosol generator to adjust a rate at which the aerosol is generated based on the indication of occupancy.

14. The method of claim 12, further comprising:
receiving measurements of a humidity level in the conditioned space; and
controlling the aerosol generator to adjust a rate at which the aerosol is generated based on the humidity level measurements.

15. The method of claim 12, further comprising:
receiving measurements of a humidity level in the conditioned space; and
controlling the aerosol generator to adjust a particle size of the aerosol that is generated based on the humidity level measurements.

16. The method of claim 12, wherein dispensing the aromatic includes dispensing two or more constituent aromatics that are individually combined with the conditioned air.

17. The method of claim 12, wherein dispensing the aromatic includes dispensing two or more constituent aromatics that are collectively combined with the conditioned air.

18. The method of claim 12, wherein the aromatic is a water soluble tablet.

19. The method of claim 12, wherein the dispensing the aromatic includes dispensing the aromatic into the condensate to produce an aromatic condensate, and energizing the condensate includes energizing the aromatic condensate to generate an aromatic aerosol including the negative ions.

20. The method of claim 12, wherein the dispensing the aromatic further includes energizing the aromatic at an aromatic aerosol generator to generate an aromatic aerosol that is combined with the conditioned air.
